(19) United States
(12) Patent Application Publication
(10) Pub. No.: US 2002/0133141 A1
(43) Pub. Date: Sep. 19, 2002

(54) Instrument shaft

(76) Inventors: Kurt D. Sparks, Palo Alto, CA (US); David J. Kupiecki, San Francisco, CA (US); Kent D. Dell, Redwood city, CA (US)

Correspondence Address: PERKINS COIE LLP, P.O. BOX 2168, MENLO PARK, CA, 94026, US

(21) Appl. No.: 09/812,355

(22) Filed: Mar. 19, 2001

(51) Int. Cl. A61M 25/00

(52) U.S. Cl. 604/523

(57) ABSTRACT

An instrument shaft for use in small openings where enhanced longitudinal dimensional stability and compressive loading characteristics are desirable. Various embodiments may be shafts of catheters used to treat peripheral vasculatures or coronary vasculatures. Embodiments of the catheter shaft provide enhanced longitudinal dimensional stability and compressive loading characteristics, while maintaining flexibility under loading conditions. In one embodiment, a catheter shaft includes an outer catheter shaft with a lumen therethrough, and an annular compression spring inside the outer catheter shaft. Also included is an actuation member inside the annular compression spring, wherein the actuation member actuates a working element at a distal end of the catheter.

### FIELD OF THE INVENTION

The invention is in the field of medical instruments and devices, and more particularly in the field of catheters.

### BACKGROUND OF THE INVENTION

Medical instruments must often be introduced into small body openings and lumens. In many instances, a small lumen must be navigated by an instrument to a site to be operated on. In such instances, it is necessary for the shaft attached to the working element to be steerable and to have the appropriate flexibility and strength. An example of a procedure in which characteristics of the instrument shaft are important is treatment of a vascular occlusion using a catheter device with some working element at its distal end.

Prior catheters used to treat near total or total occlusions have several disadvantages. For example, some catheters are not sufficiently steerable, creating a risk that the lumen will be punctured in attempting to reach or traverse the occlusion. The path through the lumen is often complex and tortuous. The occlusion itself often is irregularly shaped. Thus, a steerable catheter is required. Another disadvantage of prior catheters is that they do not have appropriate mechanical characteristics to navigate through a near total or total occlusion, particularly if the occlusion is a hard material. The catheter should have sufficient torque and compression characteristics to allow it to be twisted and pushed as necessary, for steering and to overcome resistance, without deforming undesirably.

### SUMMARY OF THE DISCLOSURE

An instrument shaft for use in various types of medical instruments is described. Various embodiments may be shafts of catheters used to treat peripheral vasculatures or coronary vasculatures. Catheter shaft embodiments provide enhanced longitudinal dimensional stability and compressive loading characteristics, while maintaining flexibility under loading conditions. In one embodiment, a catheter shaft includes an outer catheter shaft with a lumen therethrough, and an annular compression spring inside the outer catheter shaft. Also included is an actuation member inside the annular compression spring, wherein the actuation member actuates a working element at a distal end of the catheter.

### BRIEF DESCRIPTION OF THE DRAWINGS

FIG. 1 is a cross section view of an embodiment of a catheter shaft.

FIG. 2A is a cross section view of an embodiment of a compression spring.

FIG. 2B is a cross section view of an embodiment of a compression spring.

FIG. 2C is a cross section view of an embodiment of a compression spring.

FIG. 3 is a cross section view of an embodiment of a catheter shaft.

FIG. 4 is a cross section view of an embodiment of a catheter shaft.

### DETAILED DESCRIPTION

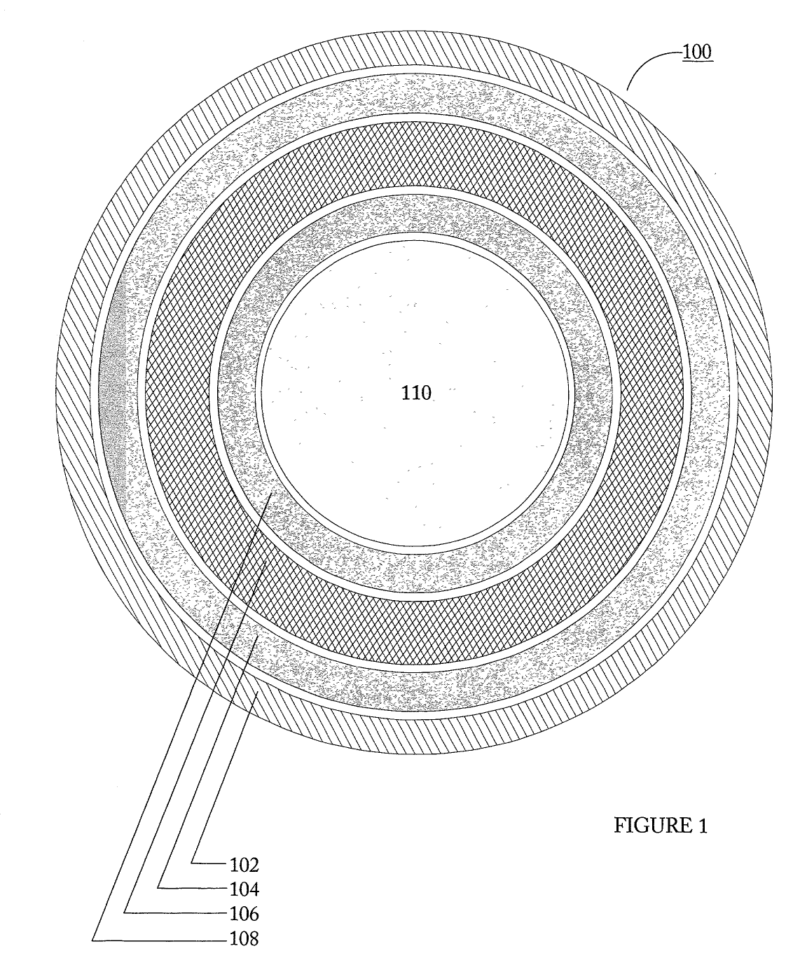

An instrument shaft is described. The instrument shaft is for any use in small openings where enhanced longitudinal dimensional stability and compressive loading characteristics are desirable. In one embodiment, the shaft is used as a catheter shaft. FIG. 1 is a cross section view of an embodiment of a catheter shaft 100 with various coaxial elements. The catheter shaft 100 may be used in catheters of any kind, but is particularly useful in situations in which enhanced longitudinal dimensional stability and compressive loading characteristics, as well as flexibility under loading are required. For example, the catheter shaft 100 is useful in situations in which the catheter must navigate a long and tortuous vasculature. The catheter shaft 100 may constitute the entire shaft of a catheter, or some portion of the shaft of the catheter.

The outer catheter shaft 102 forms an outer surface of the catheter shaft 100 and has a lumen through its entire length. The outer catheter shaft 102 may be manufactured using braided 0.0007 inch×0.003 inch stainless steel wire. In other embodiments, the outer catheter shaft 102 may be manufactured using 0.0015 inch round wire. In any case, the stainless steel wire may be wound using a known commercial braiding machine and technique. The outer catheter shaft 102 may be impregnated with a polymer material such as nylon, Pebax, Tecoflex, polyamide, or Grilamid. The durometer of the polymer with which the outer catheter shaft 102 is impregnated may be graduated in decreasing hardness toward the distal end of the catheter shaft to enhance catheter tracking characteristics through the vasculature. As used herein, “distal” refers to the direction or portion of a catheter away from an operator of the catheter and toward a working element of the catheter. As used herein “proximal” refers to the direction or portion of the catheter toward an operator of the catheter and away from the working element of the catheter.

An outer compression spring liner 104 is inside the lumen of the outer catheter shaft 102. The outer compression spring liner 104 also has a lumen through it. The outer compression spring liner 104 may be manufactured of high density polyethylene, low density polyethylene, polyamide, PTFE, or materials with similar characteristics. The outer compression spring liner 104 provides lateral support to the outer diameter of the annular compression spring 106. The support provided by the outer compression spring liner 104 reduces “walking” of filers in the annular compression spring 106 relative to each other. This helps maintain a continuous and uniform outer diameter and inner diameter of the annular compression spring 106, so that it can withstand the required compressive loading.

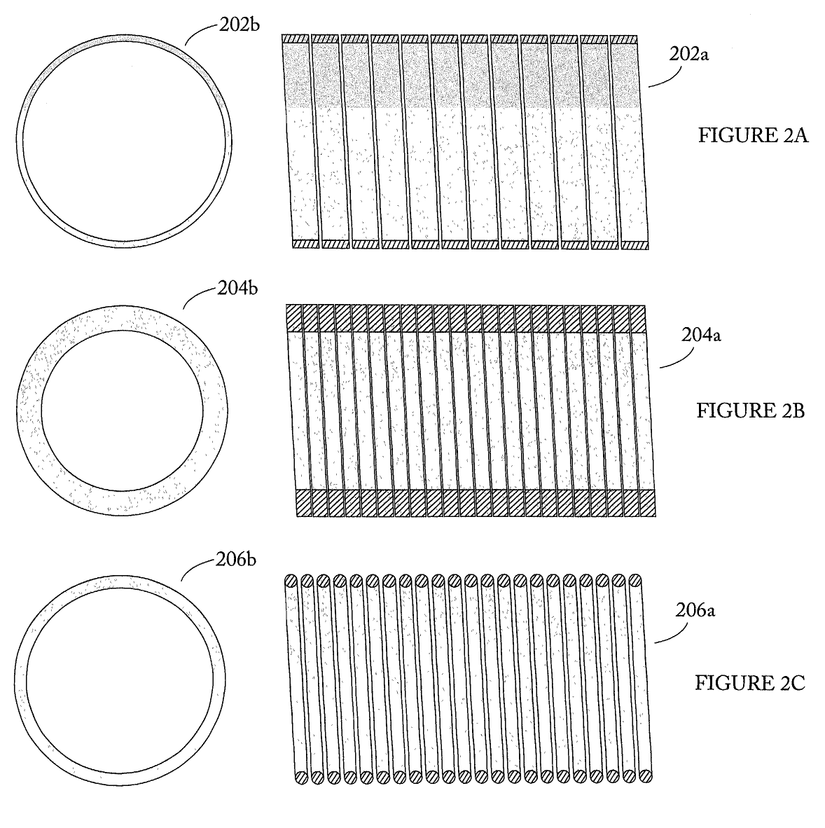

The annular compression spring 106 is inside the lumen of the outer compression spring liner 104. FIGS. 2A, 2B, and 2C show representative examples of springs that may be used as annular compression spring 106.

FIG. 2A shows two views of a flat wound spring 202. 202a is a cross section taken along the longitudinal axis of the spring 202. 202b is a cross section corresponding to a plane perpendicular to the longitudinal axis of the spring 202. Spring 202 is made of relatively flat wire, wound so as to lie flat along the outer and inner circumferences of adjoining coaxial elements of the catheter shaft 100. The support shaft 202 provides improved column loading characteristics and flexibility.

FIG. 2B shows two views of an enhanced flexibility spring 204 with a high aspect ratio winding. 204a is a cross section taken along the longitudinal axis of the spring 204. 204b is a cross section corresponding to a plane perpendicular to the longitudinal axis of the spring 204. Spring 204 is wound so that, as shown in cross section, the flat section of the wire lays against successive filers. This type of configuration provides a higher degree of column loading, in part because of the larger contacting surface areas of adjacent filers, which are normal to the column force.

FIG. 2C shows two views of an enhanced flexibility spring 206. 206a is a cross section taken along the longitudinal axis of the spring 206. 206b is a cross section corresponding to a plane perpendicular to the longitudinal axis of the spring 206. Spring 206 is manufactured using round wire, which provides a greater degree of overall flexibility.

Referring again to FIG. 1, inner compression spring liner 108 inside the annular compression spring 106. The inner compression spring liner 108 has a lumen therethrough, and may be manufactured using a variety of materials, such as polymers to provide a smooth, continuous surface for the movement of an actuation element 110. The inner compression spring liner 108 may be manufactured of high density polyethylene, low density polyethylene, polyamide, PTFE, or materials with similar characteristics. The actuation element 110 may be an actuation wire connected to a working element at a distal end of the catheter. The actuation element 110 may be also be a cable or other mechanism that generally operates by sliding proximally and distally in the lumen of the inner compression spring liner 108.

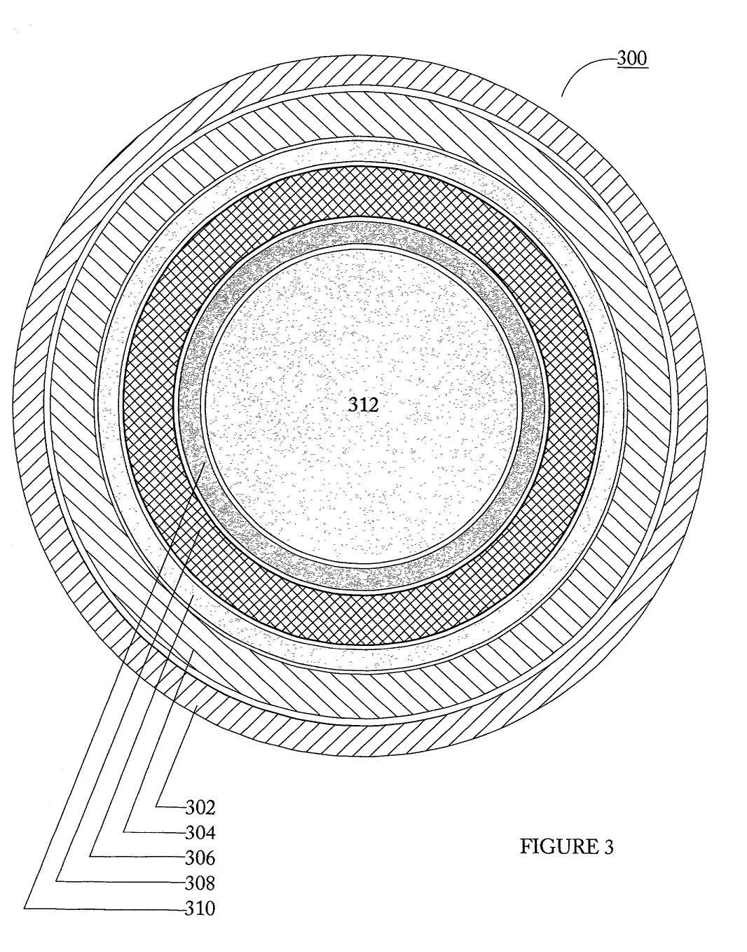

FIG. 3 is a cross section view of an embodiment of a catheter shaft 300 with various coaxial elements. Catheter shaft 300 includes some of the coaxial elements found in the catheter shaft 100 and some additional coaxial elements. The catheter shaft 300 may be used in catheters of any kind, but is particularly useful in situations in which enhanced longitudinal dimensional stability and compressive loading characteristics, as well as flexibility under loading are required. Additionally, this embodiment affords a higher degree of internal support when the shaft is acutely bent or flexed, in part because of the presence of the outer support shaft or spring 304. For example, the catheter shaft 300 is useful in situations in which the catheter must navigate a long and tortuous vasculature. The catheter shaft 300 may constitute the entire shaft of a catheter, or some portion of the shaft of the catheter.

The outer catheter shaft 302 forms the outer diameter of the catheter shaft 300 and includes a lumen therethrough. The outer catheter shaft 302 may be generally described with reference to the description of the outer catheter shaft 102 of FIG. 1. The outer support shaft or spring 304 is positioned inside the lumen of the outer catheter shaft 302 and has a lumen itself The outer support shaft or spring 304 may be manufactured as described with reference to FIGS. 2A, 2B, and 2C. Alternatively, the outer support shaft or spring 304 may be manufactured using a polymer, such as nylon, high density polyethylene, or any polymer with good mechanical properties.

The catheter shaft 300 further includes an outer compression spring liner 306, an annular compression spring 308, and an inner compression spring liner 310. The descriptions of the outer compression spring liner 104, the annular compression spring 106, and the inner compression spring liner 108 (with reference to the discussion of FIG. 1) are applicable respectively.

Element 312 is an actuation wire or cable connected to a working element at a distal end of the catheter. The element 312 may be also be a cable or other mechanism that generally operates by sliding proximally and distally in the lumen of the inner compression spring liner 310.

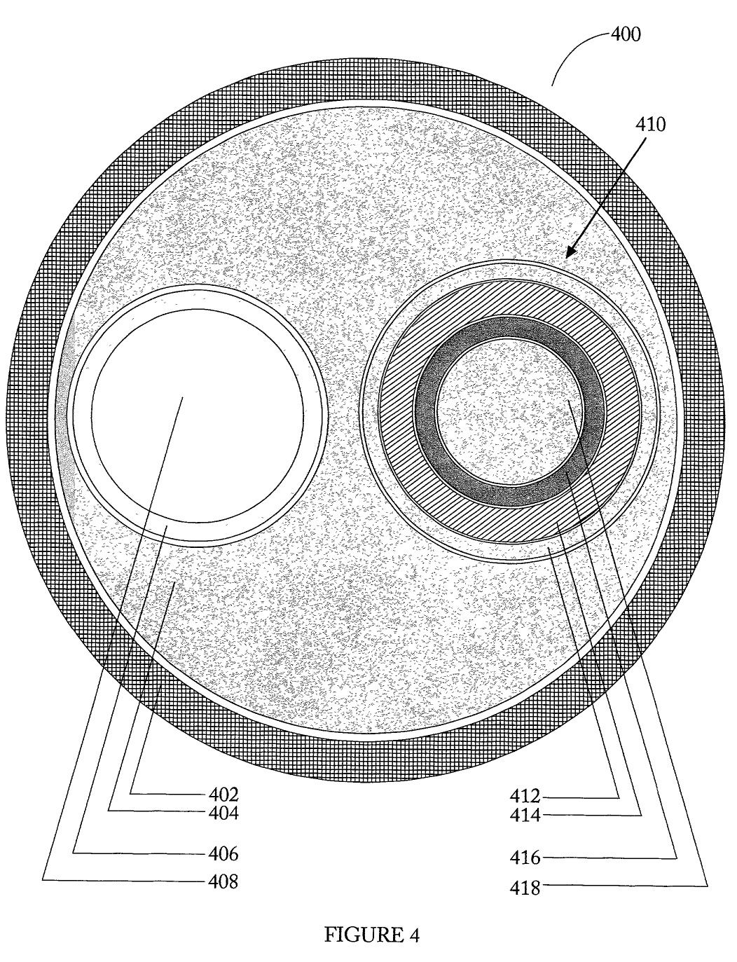

FIG. 4 is a cross section view of an embodiment of a catheter shaft 400 that includes coaxial elements arranged along more than one interior longitudinal axis. The catheter shaft 400 may be used in catheters of any kind, but is particularly useful in situations in which enhanced longitudinal dimensional stability and compressive loading characteristics, as well as flexibility under loading are required. For example, the catheter shaft 400 is useful in situations in which the catheter must navigate a long and tortuous vasculature. The catheter shaft 400 may constitute the entire shaft of a catheter, or some portion of the shaft of the catheter.

The catheter shaft 400 has an outer catheter shaft 402. The outer catheter shaft 402 forms an outer diameter of the catheter shaft 400 and has a lumen through it. The outer catheter shaft may be described with reference to the description of the outer catheter shaft 102 of FIG. 1. Inside the lumen of the outer catheter shaft is multi-lumen inner body 404. Multi-lumen inner body 404 defines two working lumens for the catheter shaft 400. In other embodiments, the multi-lumen inner body 404 defines various numbers of working lumens other than two. The catheter shaft 400 includes lumen 408 and lumen 410. Lumen 408 can be used as a guidewire lumen, or to carry fluids, while lumen 410 can be used as an actuation element lumen. The multi-lumen inner body 404 can be made of materials such as nylon or Pebax that are extruded to produce the appropriate form. Materials and durometers may be chosen to provide required attributes such as strength and flexibility for a particular application.

Lumen 408 is lined at its circumference by the guide wire liner 406. The guide wire liner 406 may be manufactured using a variety of materials, such as polymers to provide a smooth, continuous surface for the movement of a guide wire (not shown).

The lumen 410 is lined by an outer compression spring liner 412, an annular compression spring 414, an inner compression spring liner 416, and an actuation element 418. All of these items in the lumen 410 are similar to the similarly named items described with reference to FIG. 1 (elements 104, 106, 108, and 110).

Various embodiments have been described with reference to the figures, but the detailed description is not intended to be limiting. Various combinations of the elements described have not been shown, but are within the scope of the invention as defined by the claims. For example, a catheter shaft including more than two axial lumens and including some or all of the elements shown is within the scope of the claimed invention. Further, an instrument shaft manufactured using materials not specifically listed in the detailed description is within the scope of the claimed invention.

What is claimed is:

1. An instrument shaft comprising:
an outer shaft with a lumen therethrough;
an annular compression spring inside the outer shaft; and
an actuation member inside the annular compression spring, wherein the actuation member actuates a working element at a distal end of the catheter.

2. The catheter of claim 1, wherein the outer catheter shaft comprises braided stainless steel.

3. The instrument shaft of claim 2, wherein the braided stainless steel comprises braided sections of 0.0007″×0.003″ stainless steel wire.

4. The instrument shaft of claim 2, wherein the braided stainless steel comprises braided sections of 0.0015″ stainless steel round wire.

5. The instrument shaft of claim 1, wherein the braided stainless steel is impregnated with a material selected from a group comprising nylon, Pebax, Tecoflex, polyamide, and Grilamid.

6. The instrument shaft of claim 5, wherein a durometer of the material with which the braided stainless steel is impregnated decreases in hardness toward the distal end of the instrument shaft.

7. The instrument shaft of claim 1, wherein the annular compression spring is flat wound so as to have a relatively small wall thickness in cross section.

8. The instrument shaft of claim 1, wherein the annular compression spring has a high aspect ratio so as to have a relatively large wall thickness in cross section.

9. The instrument shaft of claim 1, wherein the annular compression spring is round wound wire so as to have a wall thickness approximately equal to a diameter of the round wire in cross section.

10. The instrument shaft of claim 1, wherein the actuation member comprises a wire from a proximal end of the catheter to the working element.

11. The instrument shaft of claim 1, wherein the actuation member comprises a cable from a proximal end of the catheter to the working element.

12. The instrument shaft of claim 1, wherein the actuation member comprises an extruded polymer strand from a proximal end of the catheter to the working element.

13. A catheter including a catheter shaft, the catheter shaft comprising:
an outer catheter shaft with a lumen therethrough;
an outer compression spring liner inside the outer catheter shaft;
an annular compression spring inside the outer compression spring liner;
an inner compression spring liner inside the annular compression spring; and
an actuation member inside the inner compression spring liner, wherein the actuation member actuates a working element at a distal end of the catheter.

14. The catheter of claim 13, wherein the outer catheter shaft comprises braided stainless steel.

15. The catheter of claim 14, wherein the braided stainless steel comprises braided sections of 0.0007″×0.003″ stainless steel wire.

16. The catheter of claim 14, wherein the braided stainless steel comprises braided sections of 0.0015″ stainless steel round wire.

17. The catheter of claim 13, wherein the braided stainless steel is impregnated with a material selected from a group comprising: nylon, Pebax, Tecoflex, polyamide, and Grilamid.

18. The catheter of claim 17, wherein the a durometer of the material with which the braided stainless steel is impregnated decreases in hardness toward the distal end of the catheter shaft.

19. The catheter of claim 13, wherein the annular compression spring is flat wound so as to have a relatively small wall thickness in cross section.

20. The catheter of claim 13, wherein the annular compression spring has a high aspect ratio so as to have a relatively large wall thickness in cross section.

21. The catheter of claim 13, wherein the annular compression spring is round wound wire so as to have a wall thickness approximately equal to a diameter of the round wire in cross section.

22. The catheter of claim 13, wherein the actuation member comprises a wire from a proximal end of the catheter to the working element.

23. The catheter of claim 13, wherein the actuation member comprises a cable from a proximal end of the catheter to the working element.

24. The catheter of claim 13, wherein the actuation member comprises an extruded polymer strand from a proximal end of the catheter to the working element.

25. The catheter of claim 1, wherein the outer compression spring liner is comprised of a material selected from a group comprising high density polyethylene, low density polyethylene, polyamide, and PTFE.

26. The catheter of claim 25, wherein the inner compression spring liner is comprised of a material selected from a group comprising high density polyethylene, low density polyethylene, polyamide, and PTFE.

27. The catheter of claim 1, wherein the outer catheter shaft extends an entire length of the catheter.

28. The catheter of claim 1, wherein the outer catheter shaft extends from a proximal end of the catheter and terminates short of the distal end of the catheter.

29. A catheter including a catheter shaft, the catheter shaft comprising:
an outer catheter shaft with a lumen therethrough;
an outer support shaft with a lumen therethrough, wherein the outer support shaft is in the lumen of the outer catheter shaft;
an outer compression spring liner with a lumen therethrough, wherein the outer compression spring liner is in the outer support shaft lumen;
an annular compression spring inside the outer compression spring liner lumen; and
an inner compression spring liner with a lumen therethrough, wherein the inner compression spring liner is inside the annular compression spring.

30. The catheter of claim 29, wherein the outer catheter shaft comprises braided stainless steel.

31. The catheter of claim 30, wherein the braided stainless steel comprises braided sections of 0.0007″×0.003″ stainless steel wire.

32. The catheter of claim 30, wherein the braided stainless steel comprises braided sections of 0.001″ stainless steel round wire.

33. The catheter of claim 29, wherein the braided stainless steel is impregnated with a material selected from a group comprising nylon, Pebax, Tecoflex, polyamide, and Grilamid.

34. The catheter of claim 33, wherein the a durometer of the material with which the braided stainless steel is impregnated decreases in hardness toward the distal end of the catheter shaft.

35. The catheter of claim 29, wherein the annular compression spring is flat wound so as to have a relatively small wall thickness in cross section.

36. The catheter of claim 29, wherein the annular compression spring has a high aspect ratio so as to have a relatively large wall thickness in cross section.

37. The catheter of claim 29, wherein the annular compression spring is round wound wire so as to have a wall thickness approximately equal to a diameter of the round wire in cross section.

38. The catheter of claim 29, wherein the actuation member comprises a wire from a proximal end of the catheter to the working element.

39. The catheter of claim 29, wherein the actuation member comprises a cable from a proximal end of the catheter to the working element.

40. The catheter of claim 29, wherein the actuation member comprises an extruded polymer strand from a proximal end of the catheter to the working element.

41. The catheter shaft of claim 29 wherein the outer support shaft comprises a material selected from a group comprising nylon and high density polyethylene.

42. The catheter shaft of claim 29 wherein the outer support shaft comprises a coil of flat wound wire.

43. The catheter shaft of claim 29 wherein the outer support shaft comprises a high aspect ratio coil spring.

44. The catheter shaft of claim 29 wherein the outer support shaft comprises a coil of round wound wire.

45. The catheter shaft of claim 29 wherein the outer compression spring liner is comprised of a material selected from a group comprising polyamide, nylon, and Pebax.

46. The catheter shaft of claim 29 wherein the inner compression spring liner is comprised of a material selected from a group comprising polyamide, nylon, and Pebax.

47. A catheter including a catheter shaft, the catheter shaft comprising:
an outer catheter shaft with a lumen therethrough;
an inner body in the outer catheter shaft, wherein the inner body has at least two lumens therethrough, and wherein one of the at least two lumens is a guide wire lumen and one of the at least two lumens is an actuation wire lumen; and
an annular compression spring in the actuation wire lumen, wherein an actuation wire is slidably disposed in the annular compression spring, and wherein the actuation wire controls a working element at a distal end of the catheter.

48. The catheter of claim 47, wherein the outer catheter shaft comprises braided stainless steel.

49. The catheter of claim 48, wherein the braided stainless steel comprises braided sections of 0.0007″×0.003″ stainless steel wire.

50. The catheter of claim 48, wherein the braided stainless steel comprises braided sections of 0.0015″ stainless steel round wire.

51. The catheter of claim 47, wherein the braided stainless steel is impregnated with a material selected from a group comprising nylon, Pebax, Tecoflex, polyamide, and Grilamid.

52. The catheter of claim 51, wherein the a durometer of the material with which the braided stainless steel is impregnated decreases in hardness toward the distal end of the catheter shaft.

53. The catheter of claim 47, wherein the inner body is comprised of a material selected from a group comprising extruded nylon and extruded Pebax.

54. The catheter of claim 47, further comprising an annular guide wire liner inside the guide wire lumen, wherein the guidewire liner is made of a polymer material.

55. The catheter of claim 47, further comprising an annular outer compression spring liner in the actuation wire lumen, wherein the outer compression spring liner is comprised of a material selected from a group comprising high density polyethylene, low density polyethylene, polyamide, nylon, Pebax, and PTFE, and wherein the annular compression spring is inside the outer compression spring liner.

56. The catheter of claim 47, further comprising an annular inner compression spring liner in the actuation wire lumen, wherein the inner compression spring liner is comprised of a material selected from a group comprising high density polyethylene, low density polyethylene, polyamide, nylon, Pebax, and PTFE, and wherein inner compression spring liner is the inside the annular compression spring.

57. The catheter of claim 47, wherein the annular compression spring is flat wound so as to have a relatively small wall thickness in cross section.

58. The catheter of claim 47, wherein the annular compression spring has a high aspect ratio so as to have a relatively large wall thickness in cross section.

59. The catheter of claim 47, wherein the annular compression spring is round wound wire so as to have a wall thickness approximately equal to a diameter of the round wire in cross section.

60. The catheter of claim 47, wherein the catheter shaft extends an entire length of the catheter.

61. The catheter of claim 47, wherein the catheter shaft extends from a proximal end of the catheter to the working element.
